(19) United States
(12) Patent Application Publication
(10) Pub. No.: US 2011/0230785 A1
(43) Pub. Date: Sep. 22, 2011

(54) Somatosensory Evoked Potential (SSEP) Automated Alert System

(75) Inventors: James Francis Higgins, Phoenix, AZ (US); Robert Dobos, Scottsdale, AZ (US); Norman C. Wang, Phoenix, AZ (US)

(73) Assignee: ProNerve, LLC, Broomfield, CO (US)

(21) Appl. No.: 12/724,778

(22) Filed: Mar. 16, 2010

(51) Int. Cl. A61B 5/05

(52) U.S. Cl. 600/554

(57) ABSTRACT

A processor, system, and method for alerting a surgeon to the potential of a peripheral sensory nerve injury is provided. The technology stimulates a peripheral nerve, such as the median nerve, proximate the wrist of a patient and registers the sensory evoked potential (SEP) using a cranium electrode. The SEP is analyzed to determine a slope of a portion of the SEP between the peak of the SEP and the trough of the SEP. The slope is compared to a baseline to determine whether the slope deviates a predetermined amount from the baseline slope, which is indicative of potential, imminent, or an actual positioning injury. When it is determined that the slope deviates a predetermined amount, an alarm is provided to the surgeon such that the surgeon can take corrective action.

### CLAIM OF PRIORITY UNDER 35 U.S.C. §119

None.

### CLAIM OF PRIORITY UNDER 35 U.S.C. §120

None.

### REFERENCE TO CO-PENDING APPLICATIONS FOR PATENT

None.

### BACKGROUND

1. Field

The technology of the present application relates to neurophysiology assessments during surgery, and more specifically, to measuring the rate of change of a sensory evoked potential to alert a surgeon of potential positioning injury.

2. Background

One potential complication from surgery is generally known as “positioning injury.” Intraoperative positioning nerve injuries are complications from surgery that may occur from extension or compression of nerves.

Positioning injuries are considered to be preventable although they still occur despite preventative measures. Because positioning injuries occur, some surgeries include intraoperative monitoring (“IOM”). The goal of IOM is to identify changes in brain, spinal cord, and peripheral nerve function prior to irreversible damage occurring.

IOM typically includes using an evoked potential such as, for example, somatosensory evoked potentials (SSEP), brain stem auditory evoked potentials (BAEP), motor evoked potentials (MEP), and visual evoked potentials (VEP). Electromyography (EMG) also is used extensively during operative cases. Scalp electroencephalography (EEG) provides data for analysis in SSEP, BAEP, and VEP. Scalp EEG also can be used to monitor cerebral function during carotid or other vascular surgery. In addition, EEG recorded directly from the pial surface, or electrocorticography (ECoG), is used to help determine resection margins for epilepsy surgery, and to monitor for seizures during electrical stimulation of the brain carried out while mapping cortical function.

Looking specifically at SSEP, SSEPs are recorded by stimulating peripheral afferent nerves, usually electrically; they are recorded with the help of scalp electrodes. Because of the presence of nonspecific EEG background activity, the evoked potential must be averaged to improve signal-to-noise ratio.

In intraoperative use, the median, or ulnar nerves at the wrist are the most common stimulation site for upper extremity monitoring. In the lower extremity, the posterior tibial nerve just posterior to the medial malleolus, or common peroneal at the politeal fossa are used most commonly. Other sites that can be utilized include the ulnar and peroneal nerves.

Needle electrodes generally are used to reduce artifactual signals. Recording electrodes are placed on the scalp and on the cervical spine. Additionally, electrodes can be placed at the Erb point for upper extremity SSEP recording and over the lumbosacral spine for lower extremity recording.

FIG. 1 shows a typical measure SSEP waveform 100. Waveform 100 is known as an N20 waveform and relates to the negative peak of the potential occurring at approximately 20 mseconds. The waveform 100 was generated by a peripheral nerve stimulator using a single channel constant current stimulus output applied to the wrist of a patient. The waveform 100 was the measured response by an electrode placed on the skin surface or subdermally of the patient's head. In this case, an electrode 200 was placed about 4 cm up and 2 cm back from the top of the ear 202 of the patient 204 as shown in FIG. 2.

Conventionally during IOM, amplitude, shape, and latencies of the responses are measured. Serially recorded responses are compared with baseline values. Following generally accepted IOM procedures, the patient baselines is established while the patient is under anesthesia. The patient baseline is used for a variety of reasons, but one reason in particular is due to the fact that the patient baseline may be different from the average person's due to injury or disease, and could be one of the reasons the patient is currently undergoing a procedure. Establishing a reproducible baseline recording prior to any positioning or surgical manipulations is important. Changes from the baseline responses are currently considered the most important indicators of neurological dysfunction, which is also indicative of potential, pending, or actual peripheral nerve injury and/or positioning injury. Generally, clinicians consider a 30-50% reduction in amplitude of the waveform 100 from the baseline as indicative of injury or a latency change of a 10% or 3 msecond shifts in waveform 100 from the baseline as indicative of injury. It is difficult even for trained clinicians to recognize and identify the large waveform changes identified and IOM is an expensive procedure and access to qualified technologists is extremely limited. There is, therefore, a need in the art for improved SSEP monitoring to provide automated alerts.

### SUMMARY

Embodiments disclosed herein address the above stated needs by, for example, obtaining an evoked potential waveform developed by stimulation of a peripheral nerve. The evoked potential waveform is analyzed to determine a slope over a portion of the waveform. Changes in the slope are monitored to provide an alert, warning, or indication when the change in the waveform exceeds a predefined threshold.

In one exemplary embodiment, a processor including an input port to receive a waveform from a sensor is provided. The processor is adapted to measure an evoked potential and transmit the waveform to a processing unit. The processing unit, which is coupled to the input port to receive the waveform, determines the slope of a portion of the waveform between a first point and a second point on the waveform and compares the slope to a baseline; and an output coupled to the processing unit that provides indicia based on the comparison of the slope of the portion of the waveform to the baseline.

In one exemplary methodology, a method for providing indicia to a surgeon to provide information regarding peripheral nerve injury, performed on a processor is provided. The method includes the step of generating a peripheral nerve stimulation signal and applying the peripheral nerve stimulation signal generated to a nerve to stimulate the nerve. Next, the method includes registering a sensory evoked potential waveform in response to the applied peripheral nerve stimulation. The waveform is analyzed to determine a slope of a portion of the sensory evoked potential waveform that is compared to a baseline value. Indicia may be provided to the surgeon when the comparison indicates the slope of the portion of the sensory evoked potential waveform deviates from the baseline value a predetermined amount.

In an exemplary embodiment, a processor may be specially programmed to function to provide an indication to a surgeon. The embodiment includes computer readable medium encoded with computer readable instructions for controlling the generation of peripheral nerve stimulation, measurement of an evoked response to the peripheral nerve stimulation, and analysis of the evoked response, the computer readable instructions comprising code for generating a peripheral nerve stimulation signal and code for applying the peripheral nerve stimulation signal generated to a nerve to stimulate the nerve. In response to the stimulation, code for registering a sensory evoked potential waveform in response to the applied peripheral nerve stimulation is provided. Code also is provided for determining a slope of a portion of the sensory evoked potential waveform and code is provided for comparing the slope to a baseline value wherein code provides indicia when the comparison indicates the slope of the portion of the sensory evoked potential waveform deviates from the baseline value a predetermined amount

### BRIEF DESCRIPTION OF THE DRAWINGS

FIG. 1 is an exemplary waveform relating to a sensory evoked potential (SEP);

FIG. 2 is a functional block diagram showing a system useful in measuring the SEP of a patient;

FIG. 3 is a diagram of an exemplary neurological pathway usable with the technology of the present application;

FIG. 4 is a functional block diagram of an exemplary intraoperative system consistent with the technology of the present application;

FIG. 5 is a functional block diagram of an exemplary operating system for an intraoperative monitoring system consistent with the technology of the present application;

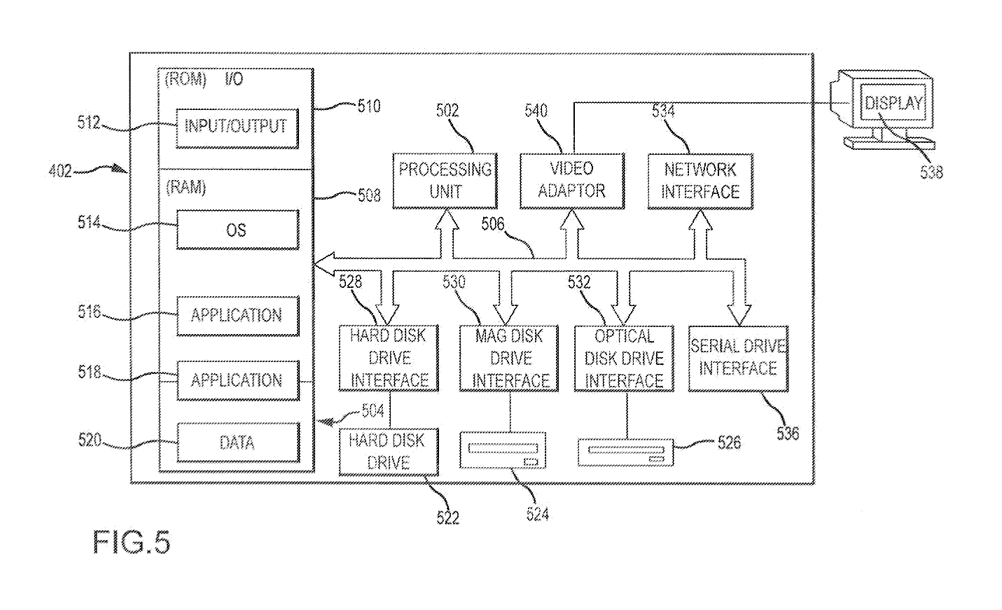

FIG. 6 is an illustrative flowchart exemplary of one method of performing operations consistent with the technology of the present application;

FIG. 7 is an exemplary waveform relating to a sensory evoked potential consistent with the technology of the present application; and

FIG. 8 is an illustrative flowchart exemplary of one method of performing operations consistent with the technology of the present application.

### DETAILED DESCRIPTION

The technology of the present application will now be described with reference to the attached figures. While the technology of the present application is described with reference to measuring a somatosensory evoked potential, one of ordinary skill in the art will recognize on reading the disclosure that other evoked potentials may be useful in relation to the technology of the present application. Moreover, the technology of the present application will be described with reference to particular exemplary embodiments. The word “exemplary” is used herein to mean “serving as an example, instance, or illustration.” Any embodiment described herein as “exemplary” is not necessarily to be construed as preferred or advantageous over other embodiments unless specifically indicated as such. Thus, the examples provided should be considered illustrative of the technology of the present application and not limiting.

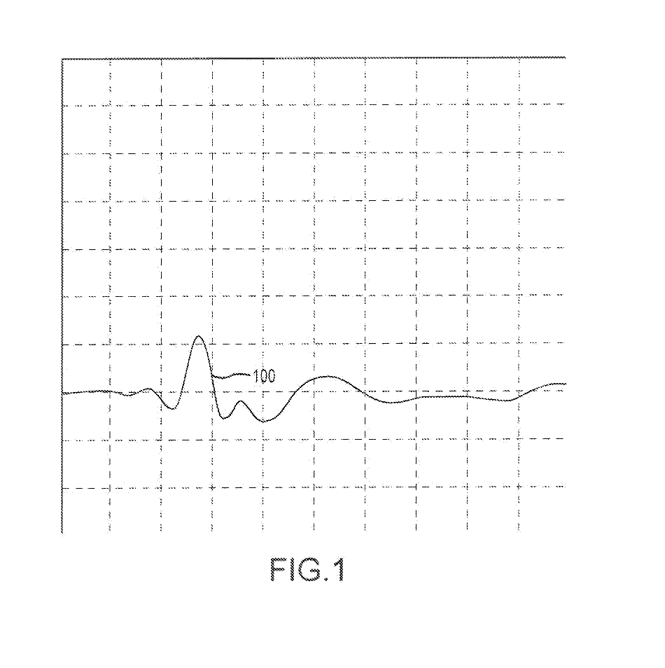

By way of background, an evoked potential is a time-locked response to a given stimulus. Generally, an electrical stimulus is provided to a muscle or nerve and a waveform, such as waveform 100 in FIG. 1, is measured in direct response to the stimulus rather than spontaneous potentials that may be measured from general brain activity. Evoked potential may be a sensory evoked potential (SEP), motor evoked potential, auditory evoked potential, visual evoked potential or the like.

Positioning injury, as generally explained above, relates to injury or damage to the neurological system between a peripheral nerve and the brain including the neurological pathway between the peripheral nerve and the brain. The pathway may include, for example, the dorsal columns of the spinal cord, the medulla of the brain stem, the thalamus, and the sensory cortex. It is believed that tactile or electrical stimulation of the peripheral nerves may provide earlier detection of potential peripheral nerve injuries and/or positioning injuries than other conventional methods. Peripheral nerve injuries as used in the present application broadly covers positioning injuries. The sensory evoked potential described in the present application is generally identified as a somatosensory evoked potential (SSEP).

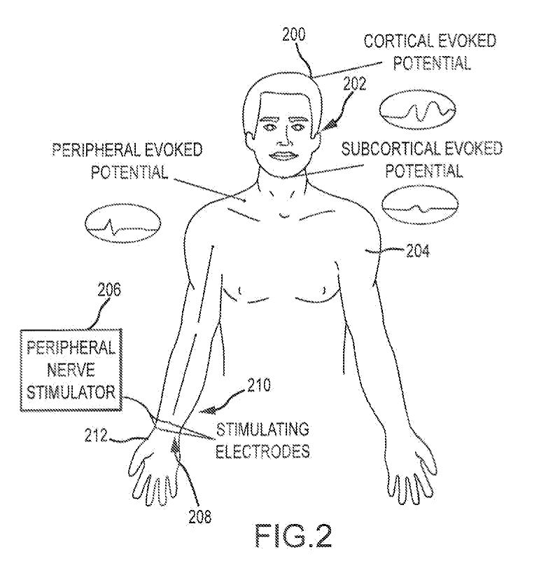

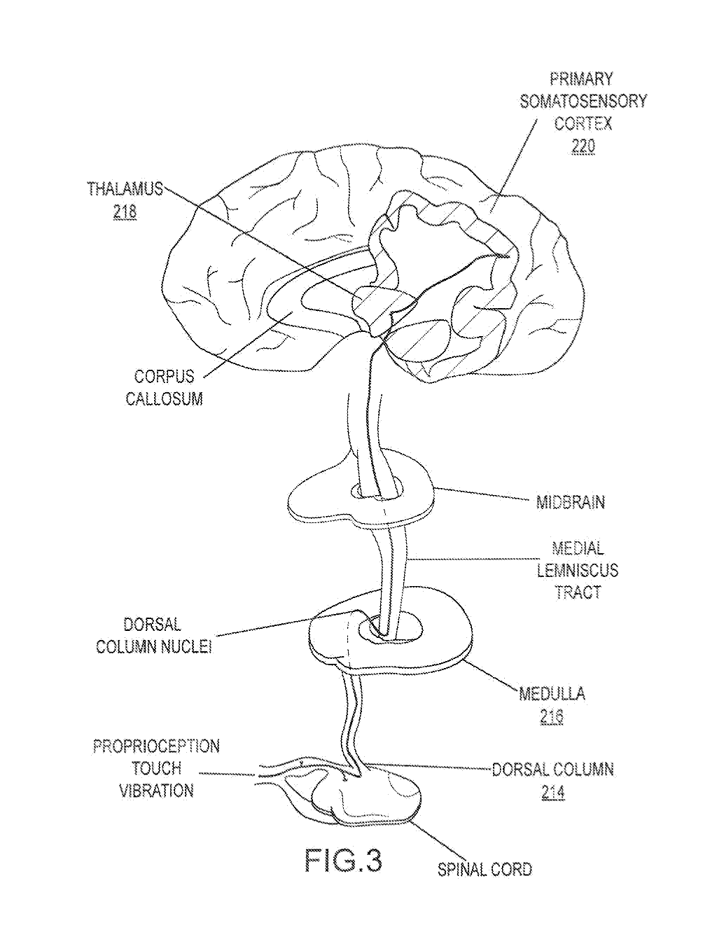

Referring back to FIG. 2, the SSEP of a patient 204 may be measured by connecting a peripheral nerve stimulator 206 to electrodes 208 attached to the patient 204. As shown, electrodes 208 are attached to the median or ulnar nerve 210 of the right wrist 212 of patient 204. The peripheral nerve stimulator 206 may be a single channel constant current stimulus output. Stimulation of the peripheral nerve may be accomplished using alternative stimulation techniques, such as, for example, movement of associated muscles, but the response to an electrical stimulation is generally easier to perform and more reliable than other methods. Referring to FIGS. 2 and 3, the electrical stimulation results in the median/ulnar nerve 210 providing a response that travels from the median nerve 210 through the dorsal column 214 of the spinal cord through the medial lemniscal pathway of the medulla 216, the ventroposteriolateral nuclei of the thalamus 218, and eventually is registered by the primary somatosensory cortex 220. The stimulation of the cortex 220 is captured by electrode 200 placed proximate the left ear 202 of the patient 204. Notice, the electrode 200 is placed by the left ear 202 because, as shown, the peripheral nerve stimulated in this example is the right wrist median nerve. If the left wrist median nerve was stimulated, the electrode 200 would be placed by the right ear. One of the benefits of SSEP is that it is not necessary to have an exact placement of the electrodes 208 and 200.

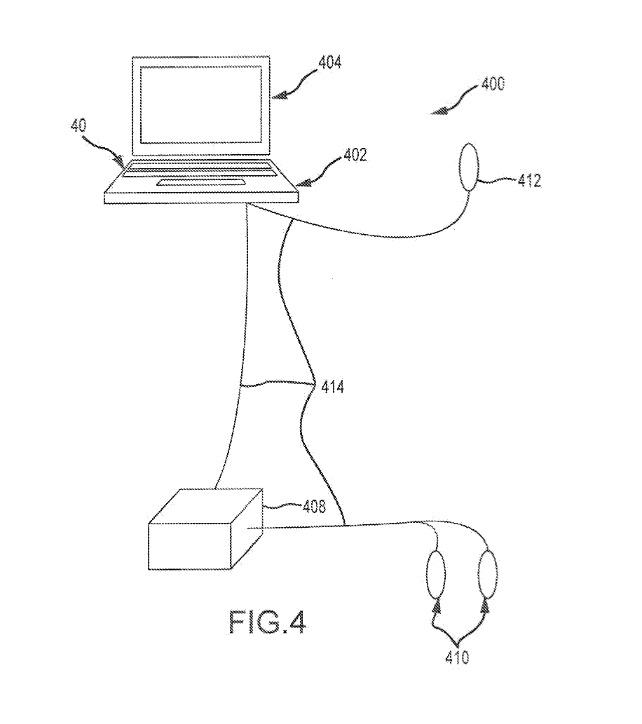

Referring now to FIG. 4, an exemplary interoperable monitoring (IOM) system 400 is shown. IOM 400 in this exemplary embodiment includes a processor 402, a monitor 404, an input mechanism 406, a peripheral nerve stimulator 408, nerve/muscle stimulation electrodes 410, cranium electrode 412, and cables 414 connecting the various equipment. One or more cables 414 may be replaced by a radio frequency transmitter and receiver as appropriate and generally known in the art.

Processor 402 may be, for example, a conventional desktop computer, a laptop computer, a patient monitoring processor, or other processing unit specially designed to perform the functions identified herein. Processor 402 will be described with reference to an exemplary operating system capable of implementing the technology of the present application. Generally, processor 402 includes a processing unit 502, a system memory 504, and a system bus 506. System bus 506 couples the various system components and allows data and control signals to be exchanged between the components. System bus 506 could operate on any number of conventional bus protocols. System memory 504 generally comprises both a random access memory (RAM) 508 and a read only memory (ROM) 510. ROM 510 generally stores a basic operating information system such as a basic input/output system (BIOS) 512. RAM 508 often contains the basic operating system (OS) 514, application software 516 and 518, and data 520. Processor 502 generally includes one or more of a hard disk drive 522, a magnetic disk drive 524, or an optical disk drive 526. The drives are connected to the bus 506 via a hard disk drive interface 528, a magnetic disk drive interface 530 and an optical disk drive interface 532. Application modules and data may be stored on a disk, such as, for example, a hard disk installed in the hard disk drive (not shown). Processor 402 also may have network connection 534 to connect to a local area network (LAN), a wireless network, an Ethernet, or the like, as well as one or more serial port interfaces 536 to connect to peripherals, such as a mouse, keyboard, modem, or printer. Processor 402 also may have USB ports or wireless components, not shown. Processor 402 typically has a display or monitor 538 connected to bus 506 through an appropriate interface, such as a video adapter 540. Monitor 538 may be used as an input mechanism using a touch screen, a light pen, or the like. One reading this disclosure, those of skill in the art, will recognize that many of the components discussed as separate units may be combined into one unit and an individual unit may be split into several different units. Further, the various functions could be contained in one personal computer or spread over several networked personal computers.

If processor 402 is connected to a network, typically one or more remote network servers exists to manage the network resources. The network server may be another computer (or processor 402 could act as the server), a server, or other equivalent device.

In operation, processor 402 would provide control signals to peripheral nerve stimulator 408 over connection 414 to generate the programmed nerve stimulator signal. The nerve stimulator signal would be applied to the nerve or muscle of the patient via stimulation electrodes 410. The nerve or muscle response would be sent over, for example in the case of the wrist, the median/ulnar nerve 210 through the dorsal column 214 of the spinal cord through the medulla 216, thalamus 218, and eventually is registered by the primary somatosensory cortex 220 where cranium electrode 412 would register the response and generate waveform 100 that would be provided to processor 402. Processor 402 would use waveform 100 to provide alerts, alarms, or warnings to surgeons regarding positioning injury.

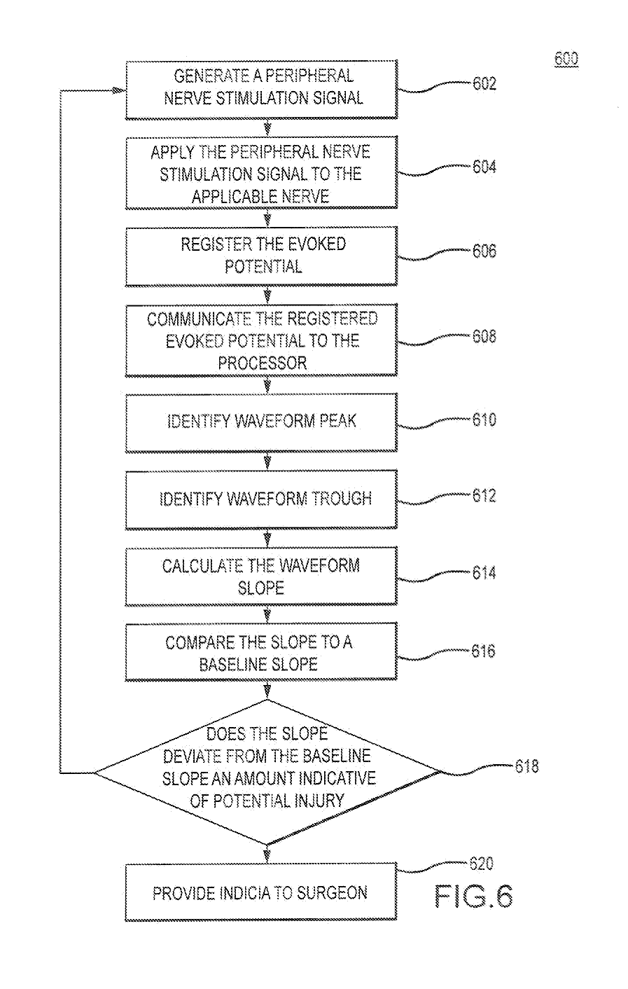

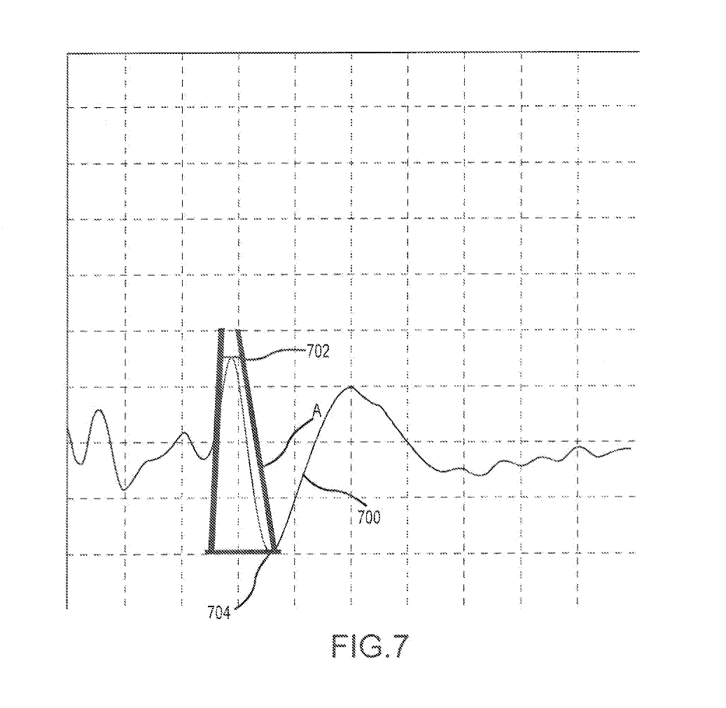

Referring now to FIG. 6, a flowchart 600 is provided with an exemplary method to identify positioning injury. While flowchart 600 is provided in certain discrete steps, one of ordinary skill in the art will recognize that the steps identified may be broken into multiple steps or multiple steps in the flowchart may be combined into a single step. Moreover, the sequence of events provided by the flowchart may be altered or rearranged without departing from the technology of the present application. With that in mind, the process begins during surgery with processor 402 providing a control signal to peripheral nerve stimulator to generate a stimulation signal, step 602. The stimulation signal is applied to the muscle or nerve via the dermal or subdermal stimulation electrodes, step 604. Alternatively to providing electrical stimulation, other forms of stimulation may be used, such as, for example, muscle manipulation via magnetic stimuli or remotely controlled, battery powered electrical devices. The stimulation results in an electrical response as identified above that is registered by the cranial electrode 412, step 606. The cranial electrode 412 communicates the response to processor 402, step 608. In the exemplary embodiment, the response may be, for example, a SSEP waveform 700 as shown in FIG. 7. The processor 402 analyzes waveform 700 and identifies the waveform peak 702, step 610, and the following waveform trough 704, step 612. Peak 702 may be point (xₚₑₐₖ, yₚₑₐₖ) and trough 704 may be point (c_{trough}, y_{trough}). Processor 402 would next calculate via a calculation module or software routine a slope A of the waveform between peak 702 and trough 704, step 614. Slope may generally be considered the rise over the run as shown by equation 1:

Slope=Δy/Δx Equation 1:

However, in the case of the waveform 700, the waveform 700 between the peak 702 and trough 704 is not a simple straight line but rather an unknown curve. Thus, processor 402 may identify a best fit curve that matches the waveform and obtain the derivative of the best fit curve to identify an approximate slope of the waveform between peak 702 and trough 704. Using a derivative, it would be appropriate to take the derivative of one or more points along the curve at any given time to identify the slope. However, as can be seen, the waveform between peak 702 and trough 704 approximates a straight line. Thus, it is believed a simple way to identify the slope of the waveform 700 between the peak 702 and the trough 704 is to obtain the secant as shown by equation 2:

Slope=(yₚₑₐₖ−y_{trough})/(xₚₑₐₖ−x_{trough}) Equation 2:

Of course, other points along the line between the peak and the trough may be used to identify the slope. While the N20 SSEP waveform may comprise approximately 600 to 700 data points, the relevant portion of the waveform for purposes of the technology of the present application may be limited to approximately 60 to 100 data points between the peak 702 and trough 704 as identified by the bracket B in FIG. 7. Moreover, it is possible other points on the waveform may similarly be used, but the slope of the waveform between the peak 702 and trough 704 appears to be the most usable portion of the waveform at present. As can be appreciated from the figures, the slope is a negative number. While the remainder of the process will be explained with reference to a negative slope, it would be rather simple to take the absolute value of the slope instead of using the real number.

The slope of waveform 700 would be compared by a comparator in processor 402 with a baseline value Z, step 616. Baseline value Z may be, for example, the average slope of the SSEP taken from an individual when not in a surgical position to get the normal SSEP waveform. Alternatively, the baseline value Z may be, for example, an average baseline across the general population, or the like. Next, it is determined whether the slope of the waveform as measured during surgery departs a predetermined amount indicative of potential injury from the baseline value Z as determined by the comparator, step 618. If it is determined that the deviation is indicative of potential, actual, or imminent injury, indicia is provided to the surgeon regarding the determination, step 620. Otherwise, the process is continually repeated until the surgery is complete.

Indicia being provided to the surgeon should be understood to generically refer to any type of alert or indication provided to the surgeon. For example, the indicia may be an audible alarm emitted from a device 416 on processor 402 such as, for example, a speaker, a buzzer, or the like. Indicia may alternatively be a visual indication on monitor 404. In another alternative, indicia may be a combination of visual and audible devices. In one particular embodiment, the visual indicia may be a real number (or an integer) where the surgeon would be trained that a value above/below a particular number indicates an alert or warning. In the case of providing only an integer, the real number would be rounded in such a manner as to provide a margin of safety, i.e., the rounding would be toward an indication that corrective action is required or desirable.

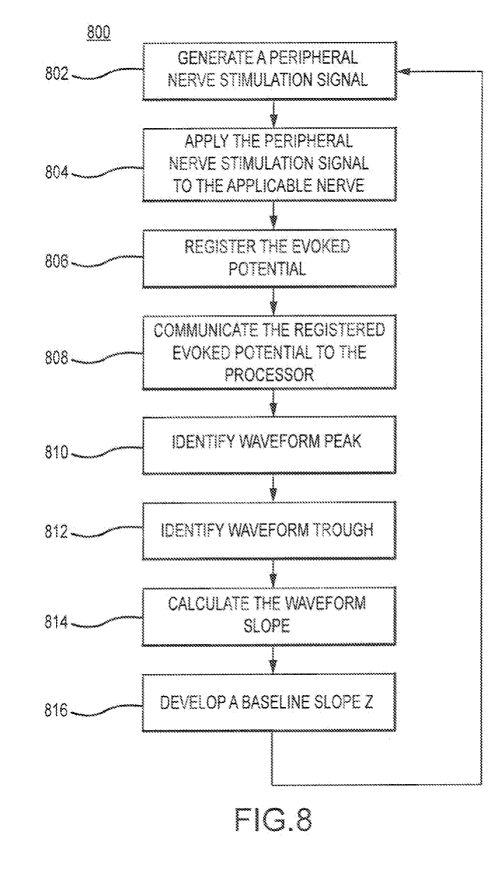

As mentioned above, the baseline slope Z may be established by a sampling of a cross section of individuals or based on exemplary SSEP waveforms. However, it would be advantageous to develop a baseline slope Z based on any particular patient's normal SSEP. Because anesthesia effects response, the baseline slope Z for any particular patient may be determined after the influence of anesthesia has be established. Referring now to FIG. 8, a flowchart 800 is provided with an exemplary method to identify a baseline slope Z. While flowchart 800 is provided in certain discrete steps, one of ordinary skill in the art will recognize that the steps identified may be broken into multiple steps or multiple steps in the flowchart may be combined into a single step. Moreover, the sequence of events provided by the flowchart may be altered or rearranged without departing from the technology of the present application. With that in mind, flowchart 800 is similar to flowchart 600 with regards to the initial steps and will not be re-explained herein for steps 802-814. Step 816, however, is used to develop a baseline slope Z for the non-surgical SSEP waveform. Thus, for each slope that is calculated, the baseline slope Z may be calculated by averaging the slopes, determining a median of the values of the slopes, using a weighted average, or the like.

Those of skill would further appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

The various illustrative logical blocks, modules, and circuits described in connection with the embodiments disclosed herein may be implemented or performed with a general purpose processor, a Digital Signal Processor (DSP), an Application Specific Integrated Circuit (ASIC), a Field Programmable Gate Array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by a processor, or in a combination of the two. A software module may reside in Random Access Memory (RAM), flash memory, Read Only Memory (ROM), Electrically Programmable ROM (EPROM), Electrically Erasable Programmable ROM (EEPROM), registers, hard disk, a removable disk, a CD-ROM, or any other form of storage medium known in the art. An exemplary storage medium is coupled to the processor such that the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A processor comprising:
an input port to receive a waveform from a sensor adapted to measure an evoked potential and transmit the waveform to a processing unit;
a processing unit coupled to the input port to receive the waveform, the processing unit determines the slope of a portion of the waveform between a first point and a second point on the waveform and compares the slope to a baseline; and
an output coupled to the processing unit that provides indicia based on the comparison of the slope of the portion of the waveform to the baseline.

2. The apparatus of claim 1, wherein indicia comprises an alarm when the comparison indicates the slope has changed by a first amount.

3. The apparatus of claim 2, wherein the indicia comprises an alert when the comparison indicates that the slope has changed between a second amount and the first amount.

4. The apparatus of claim 1, wherein the output is a monitor and the indicia is a visual alarm.

5. The apparatus of claim 1, wherein the output is a speaker and the indicia is an audible alarm.

6. The apparatus of claim 1, wherein the processor determines the slope by obtaining a derivative of the waveform between the peak and the trough.

7. The apparatus of claim 1, wherein the processor determines the slope by establishing a secant on the waveform and the slope of the secant is used to approximate the slope of the waveform.

8. The apparatus of claim 7, wherein the secant is between the peak and the trough and the slope of the portion of the waveform is the slope of the secant.

9. A method for providing indicia to a surgeon providing information regarding peripheral nerve injury, performed on a processor, comprising the steps of:
generating a peripheral nerve stimulation signal;
applying the peripheral nerve stimulation signal generated to a nerve to stimulate the nerve;
registering a sensory evoked potential waveform in response to the applied peripheral nerve stimulation;
determining a slope of a portion of the sensory evoked potential waveform;
comparing the slope of the portion of the sensory evoked potential waveform to a baseline value; and
providing indicia when the comparison indicates the slope of the portion of the sensory evoked potential waveform deviates from the baseline value a predetermined amount.

10. The method of claim 9 wherein the step of determining a slope of a portion of the sensory evoked potential waveform comprises the steps of:
identifying a peak of the sensory evoked potential waveform; and
identifying a trough of the sensory evoked potential waveform.

11. The method of claim 10, further comprising:
measuring the slope of the secant line connecting the peak and the trough of the sensory evoked potential waveform and equating the slope of the secant line with the slope of the portion of the waveform.

12. The method of claim 10, further comprising:
identifying a secant line between the peak and the trough of the sensory evoked potential waveform and equating the slope of the secant line with the slope of the portion of the waveform.

13. The method of claim 9 wherein the step of determining a slope of a portion of the sensory evoked potential waveform comprises the steps of obtaining a derivative of the sensory evoked potential waveform.

14. The method of claim 13 further comprises identifying a best fit curve that represents the sensory evoked potential waveform and determining a derivative of the best fit curve.

15. The method of claim 9 wherein the step of providing indicia when the comparison indicates the slope of the portion of the sensory evoked potential waveform deviates from the baseline value a predetermined amount comprises providing an alarm.

16. The method of claim 15 wherein the alarm is a visual alarm.

17. The method of claim 15 wherein the alarm is an audible alarm.

18. A computer readable medium encoded with computer readable instructions for controlling the generation of peripheral nerve stimulation, measurement of an evoked response to the peripheral nerve stimulation, and analysis of the evoked response, the computer readable instructions comprising:
code for generating a peripheral nerve stimulation signal;
code for applying the peripheral nerve stimulation signal generated to a nerve to stimulate the nerve;
code for registering a sensory evoked potential waveform in response to the applied peripheral nerve stimulation;
code for determining a slope of a portion of the sensory evoked potential waveform;
code for comparing the slope of the portion of the sensory evoked potential waveform to a baseline value; and
code for providing indicia when the comparison indicates the slope of the portion of the sensory evoked potential waveform deviates from the baseline value a predetermined amount.

19. A system to alert a surgeon to the potential for peripheral nerve injury comprising:
means for stimulating a peripheral nerve;
means for detecting an evoked response to the stimulation of the peripheral nerve;
means for measuring an approximate slope of the evoked response over a discrete portion of the evoked response;
means for determining whether the slope deviates from a baseline a sufficient amount to provide indicia to a surgeon; and
means for providing indicia to a surgeon that the slope deviates from a base line.

20. The system of claim 19 wherein the system further comprises a means to determine a baseline.
